(19) United States
(12) Patent Application Publication
(10) Pub. No.: US 2014/0116446 A1
(43) Pub. Date: May 1, 2014

(54) Expandable Inter Vivos Tube

(71) Applicants: Joseph A. Vilasi, Lakewood Ranch, FL (US); Joseph D'Ambrosio, Ridgefield, CT (US)

(72) Inventors: Joseph A. Vilasi, Lakewood Ranch, FL (US); Joseph D'Ambrosio, Ridgefield, CT (US)

(21) Appl. No.: 13/662,552

(22) Filed: Oct. 29, 2012

(51) Int. Cl. A61M 16/04

(52) U.S. Cl. CPC A61M 16/0434
USPC 128/207.15

(57) ABSTRACT

A flexible expandable inter vivos tube includes at least one arched segmented portion, a corresponding movable element and at least one positioning mechanism. The at least one arched segmented portion and corresponding movable element forming a flexible closed longitudinally expandable tube. The at least one arched segment includes an H-shaped connector having at least one cavity that allows variable slidable movement of a free end portion of the corresponding movable element. To move one or more of the movable elements of the expandable tube, which are within a cavity of the “H”-shaped connector, a fluid or air is introduced into the H-shaped connector running along the longitudinal axis of the flexible expandable inter vivos tube, so that the hydraulic or air pressure within an inner rib of the H-shaped connector expands the movable elements and, thus, the circumference and diameter of the flexible inter vivos tube are increased. A free end of the arched segment may include a retaining pin that engages a serrated free end of the corresponding movable element.

### CLAIM OF PRIORITY

This applicant claims, pursuant to 35 USC 119, the benefit of the earlier filing date of that provisional application entitled “Expandable Inter Vivos Tube,” filed in the US Patent and Trademark Office on Sep. 26, 2012 and afforded Ser. No. 61/705, 959, the contents of which are incorporated by reference herein.

### BACKGROUND

1. Field of the Invention

The present invention relates to the field of medical devices and, more particularly, to an expandable tube for inter vivos use in medical procedures.

2. Background of the Invention

Inter vivos tubes, such as endotracheal tubes, are used to provide gases to the lungs during surgery. For example, an endotracheal tube is inserted into the trachea with its distal tip advanced halfway toward the tracheal bifurcation to provide gases, such as oxygen and anesthetics to a patient during surgery. The exposed portion of the endotracheal tube is then firmly taped to the patient's face to prevent undesirable movement.

To align the position of conventional endotracheal tubes, an inflatable cuff balloon, at the distal end of the endotracheal tube, is inflated to correspond to the inner diameter of a portion of the trachea, thereby centering, or otherwise positioning, the endotracheal tube within the trachea. The cuff balloon, however, does not completely obstruct the entire trachea; only the portion where it is anchored is obstructed. When the cuff balloon is inflated, confirmation of the expanded balloon's contact within the trachea is achieved and delivery of anesthetic gases is performed.

Because of various sized endotracheal tubes, it is preferable to at least make the outer diameter of the endotracheal tube closely proximate to the size of the glottis, or opening between the vocal cords, for selective positioning of the endotracheal tube at a predetermined dilation. Therefore, various sized tubes are used, and the anesthesiologist or nurse anesthetist must choose from a variety of sized tubes to insert in the patient. If nasotracheal intubation or tracheostomy tubes are required in present practice even smaller interior diameters (ID) tubes are used.

Conventional endotracheal tubes vary in size and are numbered according to an internal diameter (ID). For example, for children, tubes are measured at about 3.5 to 7 mm (millimeters) internal diameter and from 7 to 11 mm for an adult. The internal diameter in women varies in general from 7.0 to 8.5 mm ID and in men from 8 to 10 mm ID. Typically, an endotracheal tube size selected for each patient is empirically selected by the anesthesiologist based on the patient's gender, age and size.

Ideally, the endotracheal tube should approximate as closely as possible the glottic size of the patient. Since there is no way to estimate the glottic size prior to the administration of anesthesia, in the existing prior art endotracheal tubes, a distal inflatable cuff is incorporated into the present day endotracheal tube which, when inflated, compresses the tracheal wall, thus creating a closed circuit between the endotracheal tube inflow from the anesthesia machine and outflow from the patient's lung to the exhalation valve. When nasotracheal intubation or tracheostomies is necessary, the internal diameter of the endotracheal tube is even less than the normal sizes, which are selected for orotracheal intubation, even greater respiratory resistance is created.

Furthermore, as noted in “Clinical Anesthesia”, 1989 Edition, J. B. Lippincott Company, edited by Paul Barash, MD, Bruce Cullen, MD, and Robert Stoelting, MD, “[e]ndotracheal tube resistance varies inversely with the tube size. Each millimeter decrease in tube size is associated with an increase in resistance of 25 ro100%. The work of breathing parallels changes in resistance. A one (1) mm decrease in tube size increases the work of breathing from 34 to 154%, depending on the ventilatory pattern”.

Therefore, in existing prior art inter vivos tubes, the internal diameter is small, and the only large portion is the external cuff balloon. This makes it harder for a surgical patient to breathe through the small internal diameter of the existing endotracheal tubes, especially if the patient must breathe spontaneously without assistance.

In summary, the prior art uses a local, inflatable balloon at the distal portion of an endotracheal tube, which narrows the patient's air way at the vocal cord level and may damage the vocal chords of the patient, if not property installed.

Applicant's prior U.S. Pat. No. 3,968,800 dated Jul. 13, 1976 and U.S. Pat. No. 4,827,925 dated May 9, 1989 describe an adjustable endotracheal tube which is complex to expand, and which does not have flexibility in being adapted to varying sized tracheas of different patients. Applicant's other prior U.S. Pat. No. 4,722,335 dated Feb. 2, 1988 discloses an expandable endotracheal tube including two overlapping curved segments, which when joined together form a closed tube. Similarly, applicant's prior U.S. Pat. No. 5,647,358, dated Jul. 15, 1997, discloses an expandable inter vivos tube that provides for expansion of the tube along at least designated parts of the tube. However, the configuration may be conceptually possible but in practical terms, difficult to construct and maintain at present prices.

Hence, there is a need in the industry for an expandable inter vivos tube that is easy to construct, easy to install, expand and remove during a procedure while reducing construction and costs of construction.

### SUMMARY OF THE INVENTION

It is therefore an object of the present invention to provide a flexible, expandable inter vivos tube that expands its internal diameter at the glottic region of the trachea, to make breathing easier for a surgical patient.

Another object of the flexible, expandable inter vivos tube of the present invention is to vary a size of the internal diameter (ID) of an endotracheal tube in order to reach the glottic size of the patient without the intervention of a distal inflatable cuff. With the present invention, the distal cuff is unnecessary and the one size endotracheal tube would fit most all adult patients. The present invention is especially useful in nasotracheal intubations where normally an even smaller internal diameter tube would be selected by the anesthesiologist.

It is also an object of the present invention to provide an endotracheal tube that maintains the same wall thickness throughout, without tapering.

It is yet another object of the present invention to provide an inter vivos tube having an internal diameter that remains substantially consistent from a proximal end to a distal end.

Another object of the present invention is to provide a vessel for administration of anesthesia by means of a flexible expandable tube that can be positioned correctly without interrupting gas flow and/or organ activity of a surgical patient.

It is also an object of the invention to provide a tube that can operate as an artificial flexible expandable vessel, such as a segment of a blood vessel to replace clogged arteries, or as a permanent catheter duct for providing fluids to or from the body.

It is also an object of the present invention to improve over the disadvantages of the existing prior art expandable tubes.

The basic concept of the present invention is to equip an inter vivos vessel, such as an endotracheal tube, artificial blood vessel or other tube with a positioning mechanism that is activated from a proximal end of the vessel and allows exact positioning and reversible anchoring within a body cavity, such as the trachea. The expandable tubes discloses herein can also be utilized as esophageal dilators, laparoscopic tubes, etc.

In the endotracheal tube embodiment, exact positioning and anchoring provide the conditions to provide anesthetic gases at the target, namely to the bronchial tubes, and ultimately the lungs.

In the present invention, the endotracheal tube can be anchored in the internal diameter of a body cavity, such as the trachea. The tube is expanded in size by means of an axially and longitudinally extendable elements inserted within the opposite free ends of a cul-de-sack formed by an H-like element. The extendable member includes free ends that run substantially the longitudinally length of the intro vivos tube. The two free edges of the extendable (flexible) cylindrical body elements engage corresponding free ends of the H-shaped element, which is curved to complete the circumference of the flexible expandable endotracheal tube. The “H” segment also provides for the integrity of the tube and, is constructed of a more rigid plastic than the rest of the tube itself. The remainder of the endotracheal tube utilizes the same or similar semi-rigid materials used in conventional inter vivos tubes. Polyvinyl tubes are presently used and continue to be used with varying degrees of hardness.

Moreover, upon extubation of the inter vivos tube of the present invention, retraction of the diameter of the tube is not required. By axially shifting the segmented arches away from each other at the free ends of the tube within the cul-de-sac of the “H” shaped element, the segmented arches are expanded so that the size of the endotracheal tube is increased and anchored during the administration of anesthesia. The segmented arches can be spread axially and longitudinally away from each other at the free ends thereof by injecting gas (or air) or fluid such as (saline) with a syringe connected to a one way valve and tube inserted in the lumen of a longitudinal canal within the rib of the “H”.

The free ends of the flexible interrupted cylindrical tube are axially and longitudinally displaced away from each other so that the internal diameter of the endotracheal tube is expanded to anchor the tube within a body cavity, such as the trachea. One or more entry points may be used to provide fluid or air within a selected longitudinally extending rib of the “H” like element. The entry point(s) are also within a canal location in the wall on the expandable tube.

The longitudinal rib within the “H” is pierced at two or more levels along the course of the “H” element in order to distribute the gas or fluid to substantially the length of the tube substantially uniformly.

It is important to note an expandable membrane is sealed to the inner and outer surfaces of the “H” element and also completely surrounds the free ends of the H-shaped element. However, the portion of the membrane that surrounds the free arms of the “H” will allow the opposite free longitudinal ends of the endotracheal tube to remain inserted within the cul-de-sac formed by the free arms of the “H” element. When air or fluid is injected into a longitudinal channel within a rib of the “H”, the two free ends of the endotracheal tube will side substantially evenly apart to a desired expansion.

In another aspect of the invention, an optional non-expandable membrane can be fused along the entire length of the outer part of the “H” element and on the two expanding arms of the endotracheal tube longitudinally at a distance away from the free arms of the “H” element equal to the depth of the cul-de-sac. In this manner the tube cannot over expand.

In another aspect of the invention, the entire endotracheal tube can, itself, be sealed by a condom—like membrane to maintain smoothness and to help maintain the integrity of the tube itself.

According to an embodiment of the invention, the free end of one side of the cylindrical body, or segmented arch, can be moved, and the opposite side would be firmly attached inside the other free end of the H-shaped element. By means of the self-acting spreading of the endotracheal tube after insertion, the position of the endotracheal tube is maintained so that controlled anesthesia can be performed without gas regurgitation.

In another embodiment of the invention, the free ends of the “H” element may include a retaining or locking point that engages saw-tooth means or serrations in the extendable elements inserted within the free ends of the cul-de-sack formed by the “H” element. The engagement of the retaining point of the free-end of the “H” element and the serrations in the extendable elements lock the extendable element in an extended position.

In this embodiment of the invention, an expandable tube (referred to as an expander tube) may be inserted into the inter vivos tube in order to expand the extendable elements of the inter vivos tube to a desired position. The expander tube may then be removed after a desired expansion of the inter vivos tube is achieved. The expander tube may be reused, if desired, after sterilization.

In another embodiment of the invention, the retaining point of the free end of the “H” element may be hinged to lock the extendable elements to remain in the expanded mode.

In one embodiment of the invention, an inter vivos system is disclosed which comprises an expandable inter vivos tube comprising: a longitudinal H-shaped member comprising: an arched outer member; an arched inner member; a rib member connecting, at a substantial midpoint of said arched outer member and said arched inner member, said arched outer member, said arched inner member and said rib member forming first and second cavities, respectively; a retaining pin positioned on a free end of one of said arched outer member and said arched inner member, said retaining pin projecting into an opening of a corresponding one of said first and second cavities, and a flexible tube split along a longitudinal axis, said split forming first and second free ends, said first and second free ends engaging corresponding ones of said first and second cavities, wherein each of said first and second free ends include at least one serration, said at least one serration engaging said retaining pin, wherein flexible tube and said arched outer member having a radius forming said inter vivos tube with a substantially circular cross-section; and an expansion means comprising: a hollow tube member including a plurality of egress points along a longitudinal axis of said tube; and an expandable member attached to said proximate end and to said distal end of said tube member; wherein said tube member is sized to fit within an inner diameter of said expandable inter vivos tube.

The inter vivos tube of the present invention, advantageously, expands substantially uniformly along its entire axial length, as fluid or air is pumped from a syringe into expansion lumens within the rib of the “H” or by the insertion of an expander tube.

### BRIEF DESCRIPTION OF THE DRAWINGS

The advantages, nature, and various additional features of the invention will appear more fully upon consideration of the illustrative embodiments to be described in detail in connection with accompanying drawings wherein like reference numerals are used to identify like element throughout the drawings:

FIG. 1 illustrates a prospective view of conventional endotracheal tube with an expanded distal cuff which compresses distally against the tracheal wail.

FIG. 2 illustrates a prospective view of a conventional tracheostomy tube inflated distally in the same manner, as in FIG. 1.

FIG. 3 illustrates a prospective view of a conventional endotracheal tube inserted through the vocal cords and expanded within the trachea.

FIGS. 4A and 4B illustrates cross-sectional views of a first and second aspect of inter vivos tubes in accordance with the principles of the invention.

FIGS. 5A and 5B illustrate prospective exploded views of inter vivos tubes in accordance with a first embodiment of the invention.

FIG. 6 illustrates a prospective view of a means for causing expansion of the inter vivos tube shown in FIGS. 4A and 4B.

FIGS. 7A and 7B illustrate prospective views of inter vivos tubes in accordance with a second embodiment of the invention.

FIG. 8 illustrates a prospective view of an inter vivos tube in accordance with a third embodiment of the invention.

FIGS. 9A and 9B illustrate a cross-sectional view and an expanded cross-sectional view, respectively, of the embodiment of the inter vivos tube shown in FIG. 8.

FIG. 10 illustrates a cross-sectional view of another aspect of the inter vivos tube shown in FIG. 8.

FIG. 11 illustrates a first mode of an expansion means for expanding the inter vivos tube shown in FIG. 8.

FIG. 12 illustrates an expandable inter vivos tube in an expanded mode including expansion means shown in FIG. 11.

FIGS. 13A and 13B illustrate a cross sectional views of the inter vivos tube including the expansion means in accordance with the principles of the invention.

It is noted that the drawings of the invention are not to scale. The drawings are intended to depict only typical aspects of the invention, and therefore should not be considered as limiting the scope of the invention. In the drawings, like numbering represents like elements between the drawings.

It is to be understood that the figures and descriptions of the present invention described herein have been simplified to illustrate the elements that are relevant for a clear understanding of the present invention, while eliminating, for purposes of clarity many other elements. However, because these elements are well-known in the art, and because they do not facilitate a better understanding of the present invention, a discussion of such element is not provided herein. The disclosure herein is directed to also variations and modifications known to those skilled in the art.

### DETAILED DESCRIPTION OF THE PRESENT INVENTION

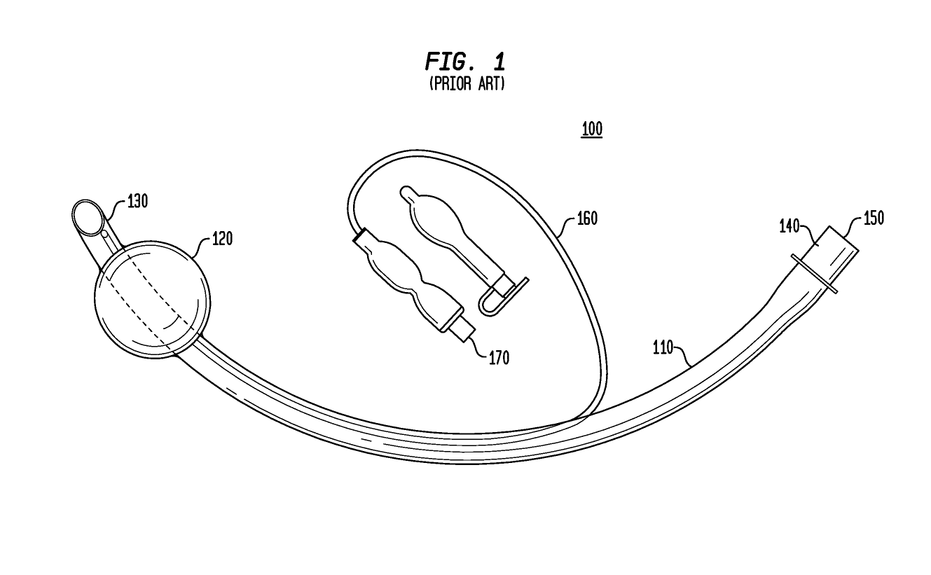

FIG. 1 illustrates a conventional endotracheal tube (i.e., inter vivos tube) 100 represented as an elongated tube 110 having a bulb member 120 positioned on a distal end 130 and a connection member 140 on a proximate end 150. The connection member 140 on proximate end 150 provides a means for allowing gases to flow through inter vivos tube 100 to distal end 130. Bulb member 120, which is shown in an expanded position, seals a passageway (not shown) into which inter vivos tube 100 is inserted to prevent gases exiting the distal end 130 from escaping along the inter vivos tube 100.

FIG. 1 further illustrates a smaller tube 160 running along an inner edge of inter vivos tube 100. Tube 160 may be used to provide a fluid, e.g., air or liquid, to bulb member 120 so as to expand bulb member 120 to the illustrated inflated position. Tube 160 may be connected to an air or liquid supply (not shown) by connection member 170.

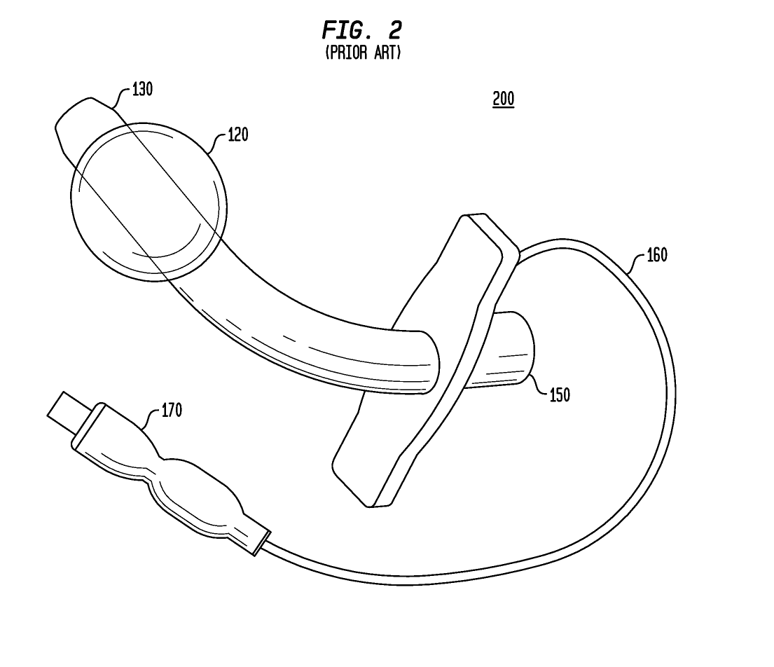

FIG. 2 illustrates a conventional tracheostomy tube (i.e., inter vivos tube) 200 used in providing air to a patient undergoing a tracheostomy process. Inter vivos tube 200 operates in a manner similar to that of the inter vivos tube 100 shown in FIG. 1, wherein a bulb member 120, positioned at a distal end 130, is expanded to prevent a fluid (e.g., air or liquid) injected from the proximate end 150 from escaping along the inter vivos tube 200. A fluid, such as air or liquid, enters through connection member 170 to expand bulb member 120, as previously discussed.

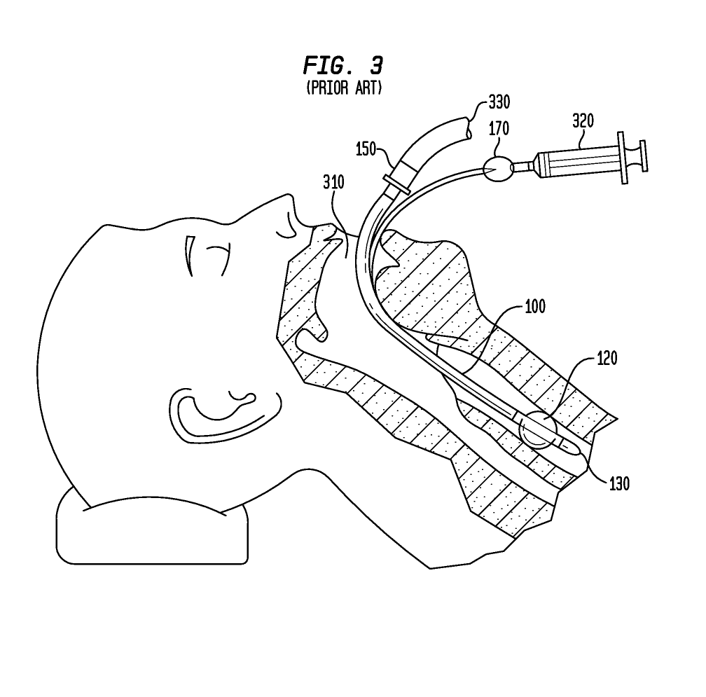

FIG. 3 illustrates a cross-sectional view 300 of the insertion and positioning of a conventional endotracheal tube 100 through a patient's vocal cords. As shown, bulb member 120 is an expanded mode to seal the patient's air passage 310. Also shown is syringe 320 that is connected to connection member 170 that represents a means for providing fluid to bulb member 120 so as to expand bulb member 120 to seal air passage 310. Also shown is tube 330 that is connected to connection member 170 to allow a fluid (e.g., gas, air, liquid) to pass from proximate end 150 of the inserted endotracheal tube 100 to distal end 130 of the inserted endotracheal tube 100.

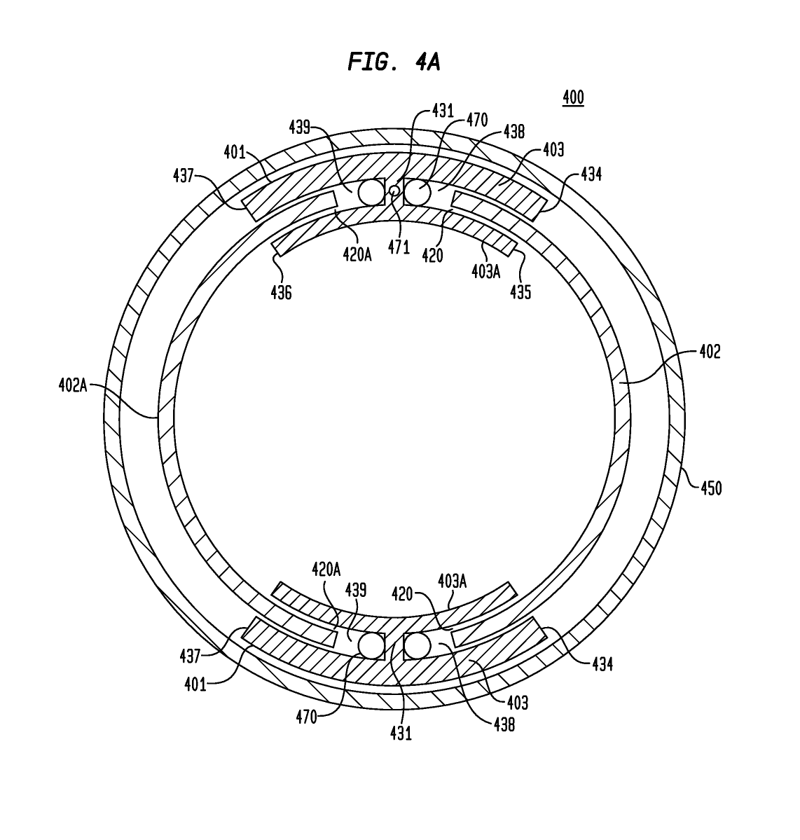

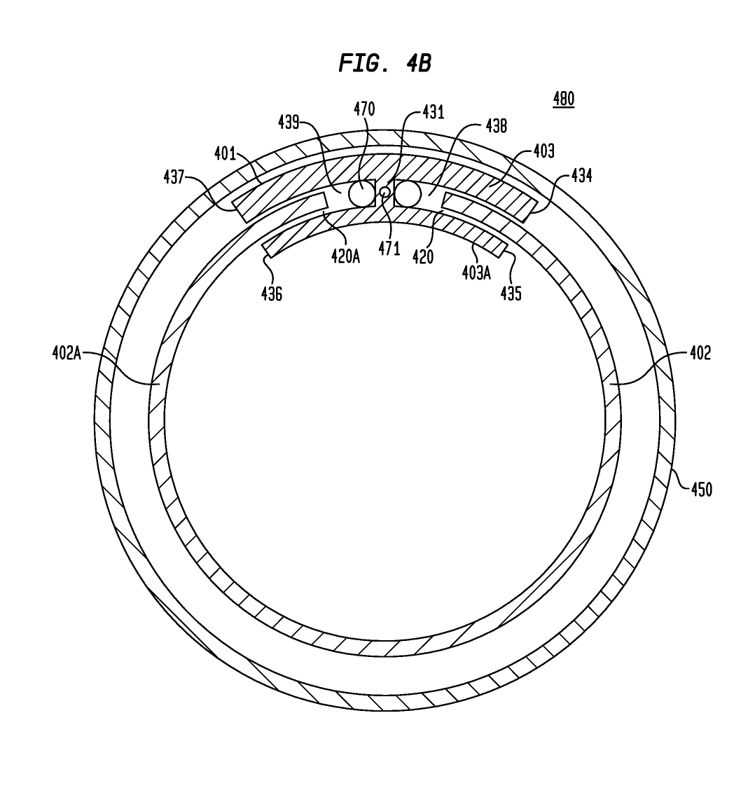

FIG. 4A illustrates a cross-sectional view of an exemplary inter vivos tube 400 in accordance with the principles of the invention. As shown, inter vivos tube 400 includes two (2) H-shaped connector elements 401 opposite to each other and extending substantially longitudinally along an edge of inter vivos tube 400. Each of the H-shaped connector elements 401 comprises an arched outer element 403 (outer circumference element) and an arched inner element 403A (inner circumference element) arranged circumferentially opposite each other at equal angles to each other along a circumference of the flexible expandable inter vivos tube 400. The H-shaped connection 401 includes rib 431, which represents the cross-bar of the “H” in the H-shaped connection member 401 joining at a substantial midpoint of the arched elements 403 and 403A. The H-shape connector elements 403 and 403A, taken with rib 431, also form cul-de-sac receptacle cavities 438 and 439, respectively. The cul-de-sac cavities 438 and 439 have an opening that is sized to receive, in tongue-in-groove-like fashion, free end tongue portions 420 and 420A of arched tube segments 402 and 402A, respectively. The H-shaped connector member 401 has respective free ends 434, 435 that define cavity 438 and free ends 436 and 437 that define cavity 439. Outer curved or ached element 403 is longer than inner curved arched element 403A to accommodate an increase in circumference.

Rib 431 connects the arched elements 403, 403A of each H-shaped connector member 401 and provides rigidity and structural integrity for the inter vivos tube 400. The rigidity of rib 431 has sufficient flexibility to enable the inter vivos tube 400 to be inserted into the trachea of the patient and to conform to the patient's airway, while retaining sufficient rigidity to permit a medical worker to position and to insert the tube 400 against anatomical resistance of the patient's throat and airway structures. Rib 431 may also include longitudinal conduit 471 for accepting a fiber optic cable for view-Oscope enablement.

The H-shaped connector member may be made of a material such as polyvinyl chloride plastic, to provide sufficient rigidity and flexibility.

Tongues 420 of arched tube elements 402, 402A are normally in a retracted position within corresponding cavities 438, 439, providing inter vivos tube 400 with a minimum diameter.

Although not shown, it would be appreciated that the diameter of inter vivos tube 400, along an axis substantially perpendicular to the arched tube elements 402, increases when tongues 420 are forced circumferentially apart by entrance of a fluid pumped into the respective cavities 438, 439. Hence, the cross sectional profile of the inter vivos tube in accordance with the principles of the invention is one of substantially circular in an unexpanded mode and of an elliptical in an expanded mode.

The increased diameter of the inter vivos tube 400, caused by the displacement of the tongue elements 420 of corresponding arched segments 402, 402A causes the passageway (FIG. 3, 310) into which the inter vivos tube 400 is inserted to become blocked, such that air may only enter or exit the passageway through the internal diameter formed by the inter vivos tube 400.

In addition, the cavities 438, 439 and tongues 420 are sized to prevent tongues 420 from expanding to a distance that would cause tongues to exit cavities 438, 439.

Also, shown is an, optional, expandable membrane 450 that surrounds inter vivos tube 400. Optional membrane 450 may be composed of a material that provides for a smooth surface of the inter vivos tube 400. The optional membrane 450 may be composed of a material such as PVC (polyvinyl chloride) that allows for a smooth entry and exit of the inter vivos tube 400 into and out of a passage way (e.g., FIG. 3, 310).

In a second exemplary aspect of the invention, flexible membranes 470 may be incorporated into cavities 438, 439. Flexible membranes 470 may expand as fluid (gas, air, liquid) is injected into H-shaped connection member 401.

FIG. 4B illustrates a second aspect of an inter vivos tube 480, which is similar to the inter vivos tube 400 shown in FIG. 4A. In this second aspect of the invention, a single H-shaped connection member 401 is incorporated into the inter vivos tube 480. As the elements of the single H-shaped member 401 shown in inter vivos tube 480 is the same as the H-shaped connector member 401 described with regard to FIG. 4A, a detailed description of H-shaped member 401 shown in FIG. 4B need not be repeated again herein.

In this exemplary second aspect, and as previously described, a diameter of the inter vivos tube 480 increases in a direction substantially perpendicular to arched segments 402, 402A as tongues 420 are displaced from cavities 438, 439 as a fluid (or air) is injected into H-shaped connector member 401, as previously described.

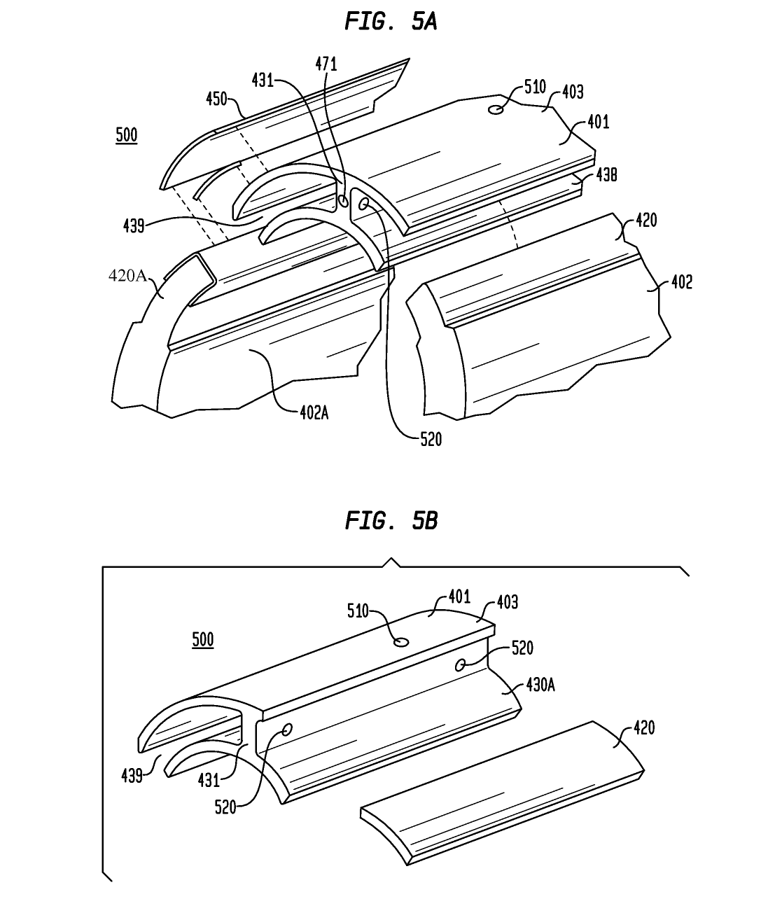

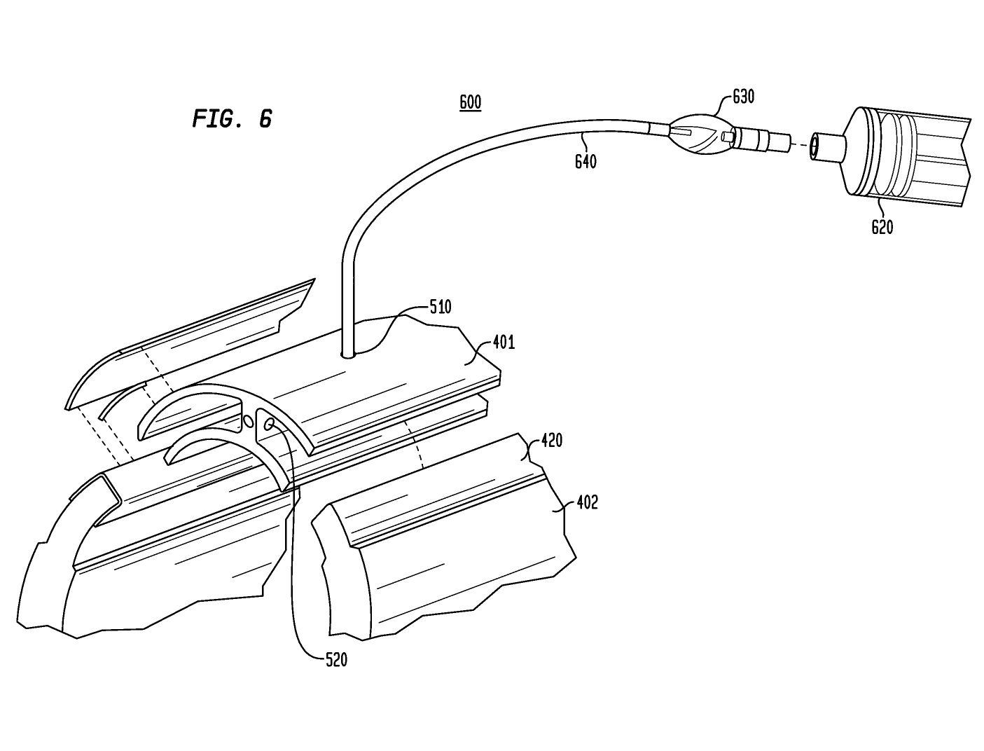

FIG. 5A illustrates an expanded prospective view of the inter vivos tube 400 shown in FIG. 4B, wherein flexible membranes 470 are not illustrated so as to illustrate a means for causing tongues 420 to be displaced from cavities 438, 439. In this exemplary embodiment shown, tongues 420 may be tapered to allow easy enter, or exit, of elements 402, 402a into cavities 438, 439, respectively.

Also illustrated in inter vivos tube 500 is insertion point 510 incorporated in an outer surface of H-shaped connector member 401. Insertion point 510 allows entry of a fluid (e.g., air, gas, saline solution, etc.) into the H-shaped connector member 401. Also shown is egress point 520 positioned within a surface of rib 431 separating upper arched segment 403 and lower arched segment 403A of the H-shaped connector 401. Although FIG. 5A illustrated egress point 520 on one side of rib 431, it would be recognized that a similar egress point 520 would exist on the not shown side of rib 431 to enable displacement of tongue 420 to expand element 402A.

In this illustrated case, fluid (or air) injected into insertion point 510 exits the egress points 520 to displace tongues 420 to increase the circumference of inter vivos tube 500 by increasing the diameter between the arched segments 402, 402A. That is, tongues 420, when displaced so as to be positioned in an expanded mode, causes the shape of inter vivos tube 500 to be oblong or elliptical rather than a substantially circular shape when tongues 420 are in an unexpanded state.

Although not shown it would be appreciated, that the insertion point 510 may be incorporated into an end portion of rib 431. In this matter, rib 431 may include a channel that extends from a proximate end to substantially near a distal end of inter vivos tube 500. The channel may be in fluid communication with each of the at least one egress points 520 to allow a fluid (e.g., air, gas, liquid) to be injected into cavities 438, 439.

Although not shown, it would be appreciated that a proximate end and a distal end of H-shaped connector member 401 may be sealed so that cavities 438, 439 may retain a fluid (e.g., air, gas, liquid) injected to cavities 438, 439. Thus, a sealing means (e.g., plugs) may be positioned at a proximate end and a distal end of cavities 438, 439. In this case, as a fluid (e.g., air, gas, liquid) is injected into injection point 510 and exits through egress points 520, cavities 438, 439 become filled with the injected fluid (e.g., air, gas, liquid) and tongues 420 are displaced from cavities 438, 439. Hence, a diameter of the inter vivos tube 500 increases as tongues 420 are displaced from cavities 438, 439.

FIG. 5B illustrates prospective view of the exemplary embodiment of the endotracheal tube 500 shown in FIG. 4A (or FIG. 4B), in accordance with the principles of the invention. In this illustrated embodiment, a plurality of egress holes 520 are shown incorporated into rib 471 in order to displace tongues 420 substantially uniformly from cavities 438, 439, respectively.

Although not shown, it would be appreciated that a proximate end and a distal end of H-shaped connector member 401 may be sealed so that cavities 438, 439 may retain a fluid injected to cavities 438, 439. Thus, as a fluid is injected into injection point 510 exits through egress points 520, cavities 438, 439 become filled with the injected fluid and tongues 420 are displaced from cavities 438, 439. Hence, a diameter of the inter vivos tube 500 increases as tongues 420 are displaced from cavities 438, 439.

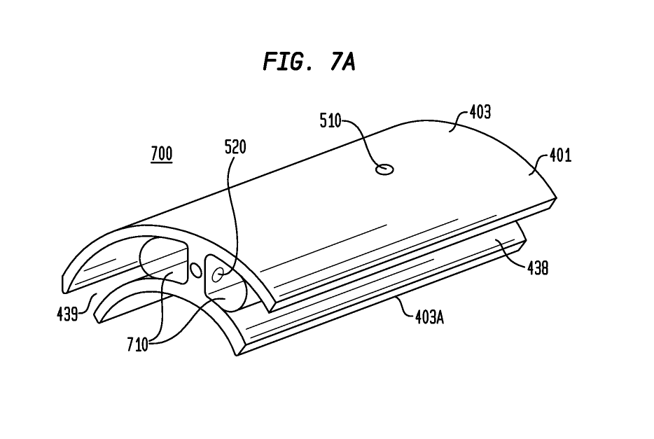

FIG. 6 illustrates prospective view of the exemplary embodiment of the endotracheal tube 600 shown in FIGS. 4A, 4B and 5A, 5B, in accordance with the principles of the invention.

In this illustrated example, a fluid, e.g., air, is injected or inserted into insertion point 510 through a syringe 620, for example. The injection process further includes a one-way value 630 that allows the fluid to pass through tube 640 into H-shaped connection member 401, through injection point 510 and exit egress points 520. The injected fluid displaces tongues 420, as previously described, to expand the diameter of the inter vivos tube 600. One way value 630 allows the fluid to pass in a first direction to displace tongues 420 and to statically retain the injected fluid until the valve is released, causing the fluid to exit through insertion point 510.

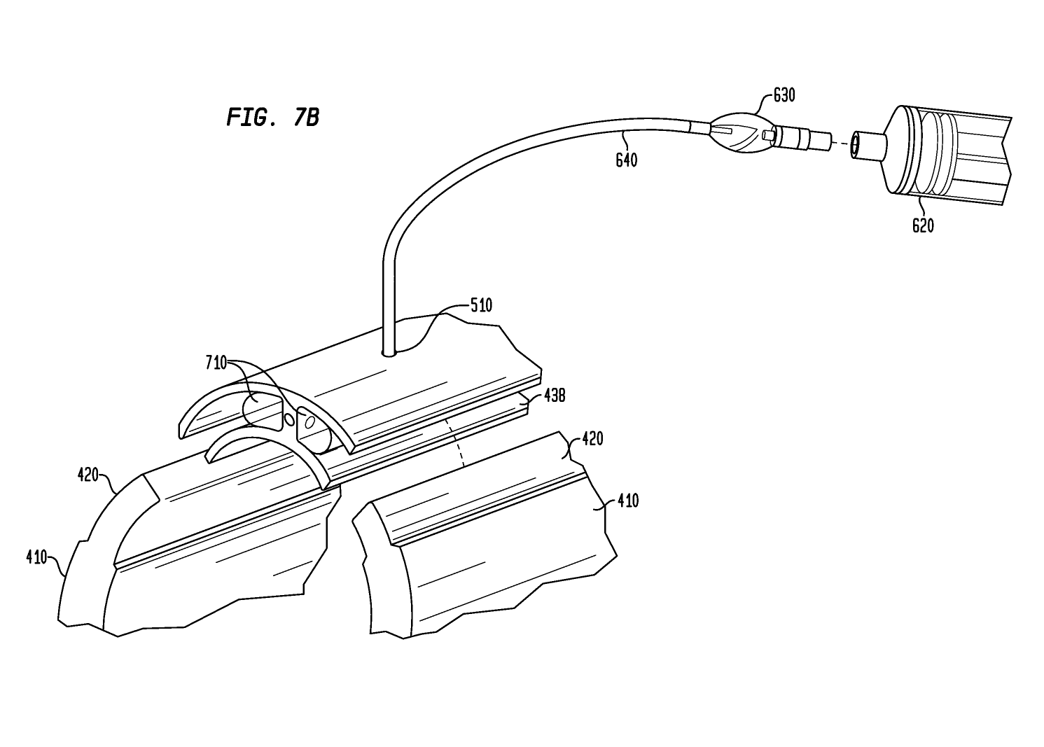

FIG. 7A illustrates a prospective view of a second aspect of an inter vivos tube 700 in accordance with the principles of the invention. In this exemplary embodiment, balloons or flexible membranes 710 are incorporated into cavities 438, 439. Flexible membranes 710 are in fluid communication with egress holes 520 (not shown) similar to those shown in FIGS. 5A, 5B, to allow fluid to be injected into flexible membrane 710. Flexible membrane 710 when filled expand to partially fill cavity 438, 439 and displace tongues 420 (not shown). Also shown, is injection hole 510. As previously discussed, injection hole 510 allows entry of an air or fluid into the ribs 431, which is ejected through egress holes 520 (not shown). In this case, flexible membranes 710 displace tongues 420 (not shown), to cause inter vivos tube 700 to increase in diameter in a direction substantially perpendicular to arched segment members 402, 402A (not shown).

FIG. 7B illustrates a second prospective view of the inter vivos tube 700 shown in FIG. 7A. In this illustrated aspect, the flexible membranes 710 operate as a means to displace the free ends or tongues 420 of arched segment elements 402, 402A from cavities 438, 439. In this case, as flexible membranes 710 expand, by the entry of a fluid through injection point 510, by syringe 620, tongues 420 are displaced from cavities 438, 439 to expand a diameter (and consequentially a circumference) of inter vivos tube 700. Similarly, as the flexible membranes 710 deflate the free ends of arched segments 402, 402A (i.e., tongue 420) may be returned to cavities 438, 439 so as to reduce the circumference of the inter vivos tube. In this case, the tongues 420 may be returned to cavities 438, 439 by means of the elasticity of the material comprising arched segments 402, 402A.

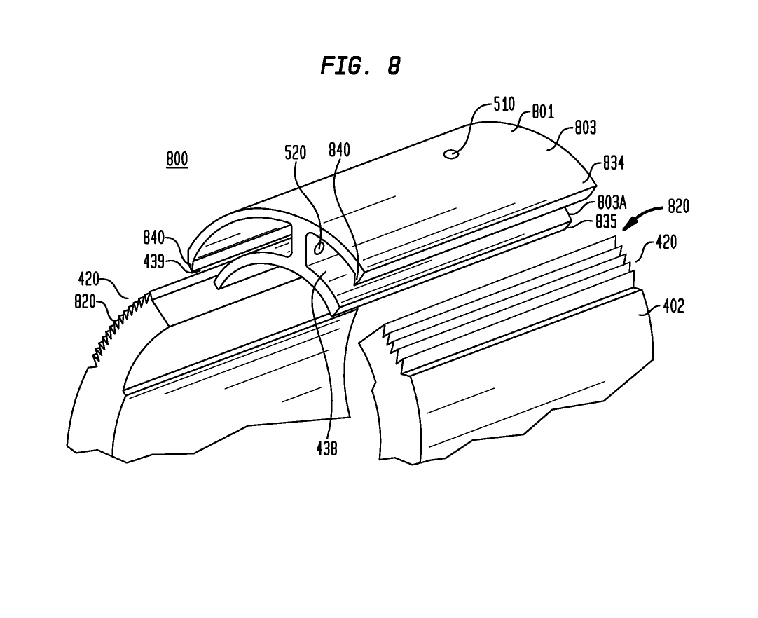

FIG. 8 illustrates a prospective view of an exemplary inter vivos tube 800 in accordance with a second embodiment of the invention. In this illustrated embodiment, H-shaped connector member 801 includes outer arched segment 803 and inner arched segment 803A, which are separated by rib 831. H-shaped connector 810 is similar to H-shaped connector described with regard to FIGS. 4A, 4B, 5A, 5B; hence, a further description of H-shaped connector membrane 801 need not be repeated, again, herein.

In accordance with this second embodiment of the invention, also illustrated, are locking pins 840 positioned on free end 834 of outer arched segment 803 extending into cavities 838, 839. Locking pins 840 restrict the opening of cavities 838, 839 and provide a means for locking tongues 420 into a desired, expanded, position, as will be described.

Also, illustrated in the inter vivos tube 800, is at least one saw tooth or serration 820 on tongue 420. The at least one serration 820 on tongue 420 are oriented in a direction to allow tongue 420 to be displaced from cavities 838, 839 and to engage locking pin 840 in order to retain tongue 420 in a desired position.

In this case, as tongues 420 are displaced from cavities 438, 439 as previously described by the addition of a fluid into cavities 838, 839, the serrations 820 engage retaining point 840 and, thus, retain tongue 420 in an extended position.

In one aspect of the invention, injection point 510 and egress point(s) 520 may be incorporated into H-shaped member 801, as previously described, to provide a means for causing tongues 420 with serrations 820 to be displaced from cavities 438, 439.

In another aspect of the invention, injection point 510 and egress point(s) 520 need not be incorporated into H-shaped connector member 810 and other means for expanding inter vivos tube 800 may be employed.

Although, locking pins 840 are illustrated as being positioned on the outer arched segment 803, it would be appreciated that locking pins 840 may be incorporated onto free end 835 of lower arched segment 803A and the serrations 420 may be positioned on a lower side of tongue 420 to engage the retaining pin 840 on the lower arched segment 803A, without altering the scope of the invention.

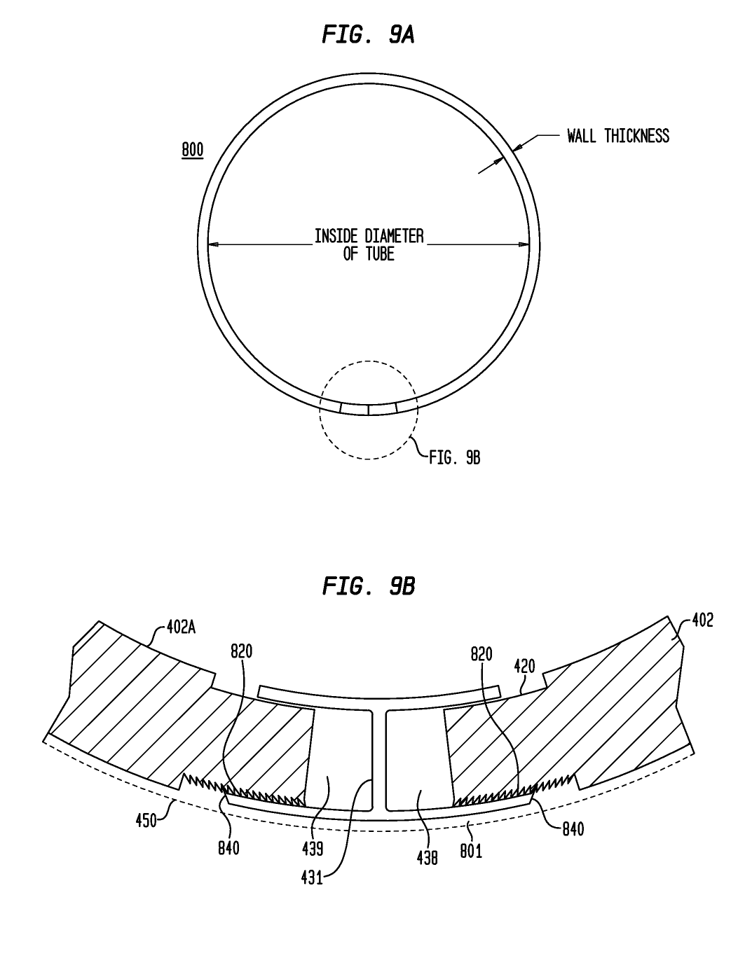

FIG. 9A illustrates a cross section view of inter vivos tube 800 having a known inside, or internal, diameter and a known wall thickness. FIG. 9B illustrates an expanded cross-sectional view of H-shaped connector 801 in accordance with the principles of the invention. In this case, tongues 420, including serrations 820 are retained in a desired position by engaging the serrations 820 into locking pin 840 as tongues 410 are displaced from cavities 438, 439. In this illustrated embodiment of the invention, as arched segments 402, 402A expand, as tongues 420 are displaced from cavities 438, 439, serrations 820 engage retaining pins 840 to create an elongated diameter substantially perpendicular to arched segments 402, 402A. Accordingly, incremental increases in the diameter of inter vivos tube 800 may be achieved by engaging different ones of the serrations 820. As would be appreciated the incremental increase in the diameter of the inter vivos tube 800 depends on the number and depth of serrations 840.

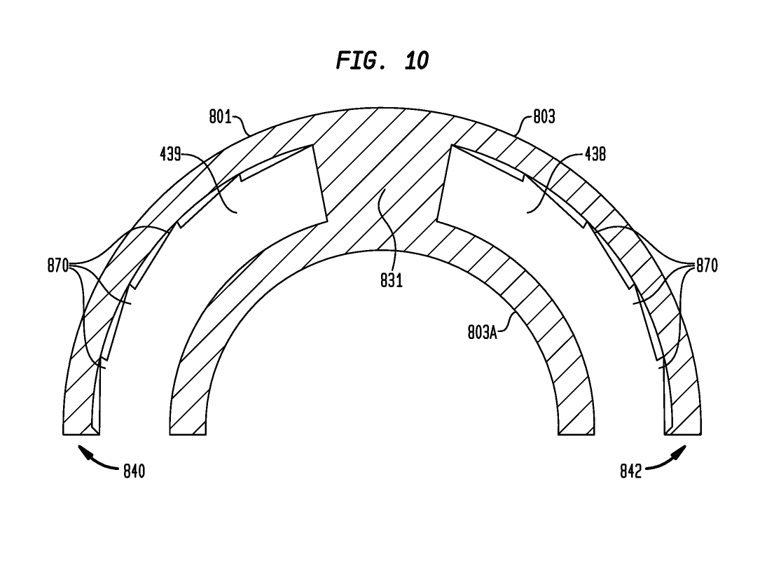

FIG. 10 illustrates an expanded cross-sectional view of an exemplary second aspect of the inter vivos tube 800 in accordance with the principles of the invention. In this second aspect of the invention, the inner surfaces of arched outer segment 803 is stepped (870) to prevent displaced tongues 420 (not shown) from being retracted into cavities 438, 439. That is, the steps 870 are oriented opposite that of serrations 820 so that the steps 870 may engage serrations 820 to prevent arched segments 402, 402A (not shown) from returning to a smaller diameter by the elastic forces of the materials from which segments 402, 402A are composed.

Also shown are retaining pins 840. In one aspect of the invention, the retaining pins 840 may be fixed, while in another aspect of the invention, the retaining pins 840 may be hinged (842) to allow easier displacement of tongues 420 (not shown) from cavities 438, 439. Hinged pins 842 provides a stop to prevent tongue 420 from being retracted into cavities 438, 439 as the hinged pin 842 swings back toward cavity 438, for example, should arched segment 402, 402A (not shown) be contracted (e.g., loss of fluid in cavities 438, 439).

That is, the use of serrations 820 is advantageous as it avoids problems that may be introduced with the inadvertent release of the means for maintaining arched segments 402, 402A in an expanded mode and, thus, causing tongues 402 to reenter cavities 438, 439.

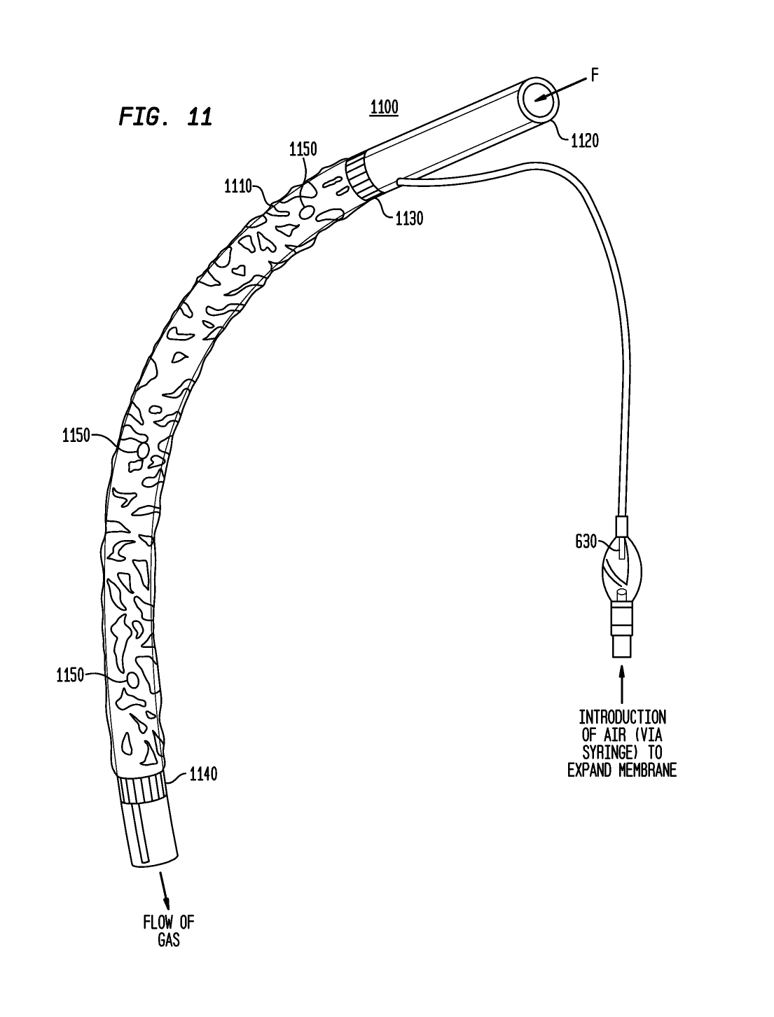

FIG. 11 illustrates an exemplary expander tube element 1100 suitable for providing a means for expanding inter vivos tube 800 in accordance with the principles of the invention.

In this illustrated extender tube element 1100, an expandable membrane 1110 surrounds a tube element 1120 and is fused to tube element 1120 at a distal end 1140 and a proximate end 1130. Within tube member 1120 are at least one egress hole 1150. Egress hole 1150, in this illustrated embodiment normally would not be visible, unless the expandable membrane 1110 is made of a clear or transparent material. However, egress holes 1150 are shown in this illustrated embodiment in order to describe the invention claimed, in sufficient detail to allow one skilled in the art to practice the invention claimed.

A means, e.g., a syringe, (not shown), allows a fluid (e.g., air, gas, liquid) to be injected into tube member 1120 through connector 630, as previously described with regard to FIG. 6. The injected fluid exits through egress holes 1150 and as the injected fluid (e.g., air, gas, liquid) is ejected through egress holes 1150, expandable membrane 1110 expands as the injected fluid fills membrane 1110. As membrane 1110 is fused to tube 1120, the fluid filling the membrane 1110 is retained within the confines of the membrane 1110.

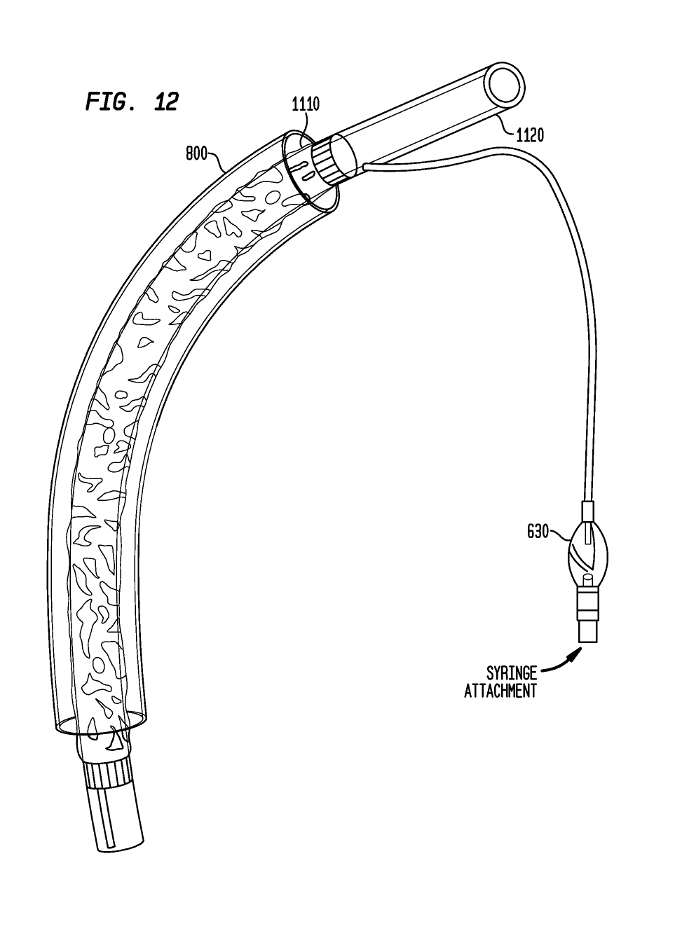

FIG. 12 illustrates an exemplary example of the insertion of expander tube 1100 into inter vivos tube 800, to cause inter vivos tube 800 to expand. In this illustrated example, arched segments 402, 402A (not shown) of inter vivos tube 800 may be expanded as expander tube 1100 is injected with a fluid, as previously described.

The use of the expander tube 1100 is advantageous, as expander tube 1100 may be deflated after the inter vivos tube 800 is expanded (and retained in position by retaining pins 840, as previously described) and the expander tube 1100 may be withdrawn from the expanded inter vivos tube 800 and re-sterilized for future use, if desired.

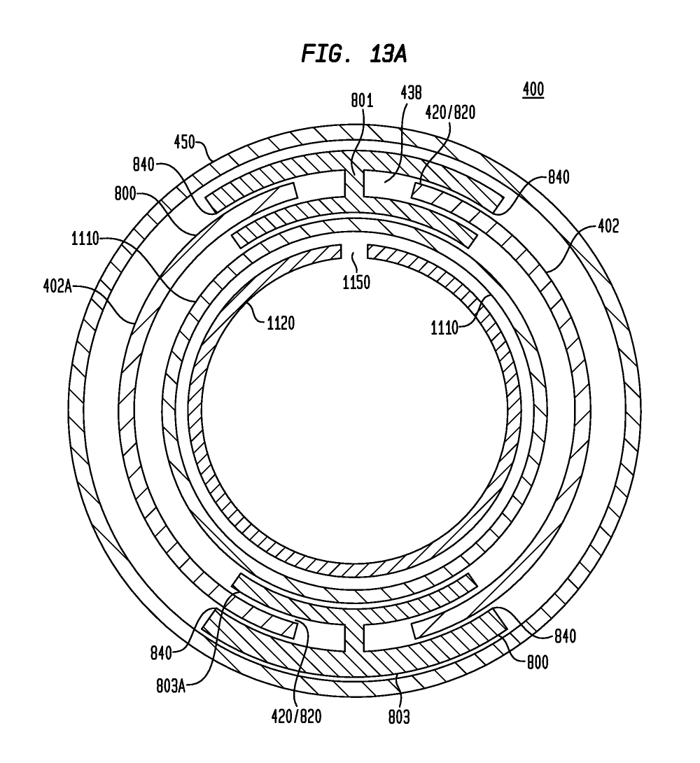

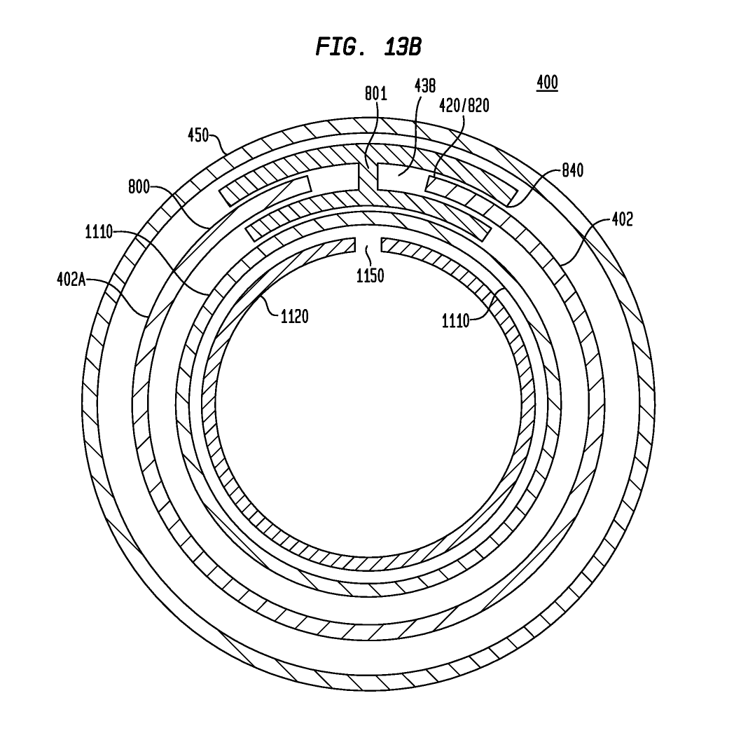

FIG. 13A illustrates a cross-sectional view of the configuration shown in FIG. 12 wherein expander tube 1100, including extendable membrane 1110 and tube 1120, are inserted within inter vivos tube 800. In this case, inter vivos tube 800 includes two H-shaped members 801 including retaining pins 840, and tongues 420 include serrations 840, as described previously.

In this illustrated case, as fluid is injected into expander tube 1120 and ejected through egress holes 1150, membrane 1110 expands as the fluid is retained in membrane 1110. As membrane 1110 expands, the expanded membrane 1110 pushes against arched segment elements 402, 402A and force tongues 420 (containing serrations 820) to be displaced from cavities 438, 439. As tongues 420 expand, serrations 820 engage retaining pins 840 (842), to retain arch segments 402, 402A, in a desired position.

FIG. 13B illustrates a second aspect of the second embodiment of the invention shown in FIG. 13A. In this case, similar to that shown in FIG. 4B, a single H-shaped connector member 801 is incorporated into inter vivos tube 800. Similar to the embodiment described with regard to FIG. 13A, as a fluid expands membrane 1110, arched segments 402, 402A extend and tongues 420 are drawn from cavities 438, 439. Tongues 420 are held in place by retain pin 840 (842) engaging serrations 820 on tongues 420, as previously described.

While there has been shown, described, and pointed out fundamental and novel features of the present invention as applied to preferred embodiments thereof, it will be understood that various omissions and substitutions and changes in the apparatus described, in the form and details of the devices disclosed, and in their operation, may be made by those skilled in the art without departing from the spirit of the present invention.

It is expressly intended that all combinations of those elements that perform substantially the same function in substantially the same way to achieve the same results are within the scope of the invention. Substitutions of elements from one described embodiment to another are also fully intended and contemplated.

The terms “a” or “an” as used herein are to describe elements and components of the invention. This is done merely for convenience and to give a general sense of the invention. The description herein should be read to include one or at least one and the singular also includes the plural unless indicated to the contrary.

The term “comprises”, “comprising”, “includes”, “including”, “as”, “having”, or any other variation thereof, are intended to cover non-exclusive inclusions. For example, a process, method, article or apparatus that comprises a list of elements is not necessarily limited to only those elements but may include other elements not expressly listed or inherent to such process, method, article, or apparatus. In addition, unless expressly stated to the contrary, the term “or” refers to an inclusive “or” and not to an exclusive “or”. For example, a condition A or B is satisfied by any one of the following: A is true (or present) and B is false (or not present); A is false (or not present) and B is true (or present); and both A and B are true (or present).

1. An expandable inter vivos tube comprising:
a first member extending longitudinally substantially along an edge of said inter vivos tube, said first member comprising:
an outer circumference member;
an inner circumference member; and
a rib element connecting said outer circumference member and said inner circumference member at substantially a midpoint of said outer circumference member and said inner circumference member, said outer circumference member, said inner circumference member and said rib member forming a first cavity and a second cavity, said first cavity being formed at an intersection of said outer circumference member, a first surface of said rib and said inner circumference member and said second cavity being formed at an intersection of said outer circumference member, a second surface of said rib and said inner circumference member;
an injection point positioned on one of said outer circumference member and an edge of said rib element; and
at least one egress point in fluid communication with said injection point, at least one of said at least one egress point positioned within each of said first surface and said second surface of said rib element facing a corresponding one of said first and second cavities; and
a flexible membrane inserted within each of said first and second cavities, said flexible membrane in fluid communication with said at least one of said at least one egress point positioned within each of said first surface and said second surface of said rib element; and
a flexible longitudinal tube member having a first free end and a second free end, said first free end and second free end slidably engaging said first and second cavities, said first and second free ends being displaced from said first and second cavities, respectively, to expand a diameter of said flexible longitudinal tube member.

2. The expandable inter vivos tube of claim 1, further comprising:
a second member extending longitudinally substantially along a second edge of said inter vivos tube, said second member, positioned opposite said first member, comprising:
a second outer circumference member;
a second inner circumference member; and
a second rib element connecting said second outer circumference member and said second inner circumference member at substantially a midpoint of said second outer circumference member and said second inner circumference member, said second outer circumference member, said second inner circumference member and said rib member forming third and fourth cavities, respectively, wherein said flexible longitudinal tube member includes a second first free end and a second second free end, said second first free end and said second second free end engaging said third and fourth cavities, respectively;
an injection point positioned on one of said second outer circumference member and an edge of said second rib element;
at least one egress point in fluid communication with said injection point, at least one of said at least one egress point positioned within each of a first surface of said second rib element and a second surface of said second rib element, said at least one of said at least egress point facing corresponding said third and fourth cavities.

3. (canceled)

4. The inter vivos tube of claim 1, further comprising:
a sealing means longitudinally positioned within said first and second cavities sealing said first and second cavities.

5. The inter vivos tube of claim 2, further comprising:
a flexible membrane, attached to each of said first surface of said second rib element and said second surface of said second rib element, said flexible membrane in fluid communication with said at least one of said at least one egress point in said first surface of said second rib element and said second surface of said second rib element.

6. The inter vivos tube of claim 1, further wherein said first free end and said second free end are tapered.

7. The inter vivos tube of claim 1, further comprising:
a retaining pin positioned at a free end of one of said outer circumference member and inner circumference member, said retaining pin positioned to restrict an opening of a corresponding one of said first cavity and said second cavity.

8. The inter vivos tube of claim 7, wherein said retaining pin is one of: fixed and hinged.

9. The inter vivos tube of claim 7, wherein said first free end and said second free end include at least one serration positioned to engage a corresponding retaining point, said serration oriented to retain said first and second free end in one of a plurality of desired positions.

10. The inter vivos tube of claim 1, further comprising:
a membrane surrounding said expandable inter vivos tube.

11. An inter vivos tube comprising:
a longitudinal H-shaped member comprising:
an arched outer member;
an arched inner member;
a rib member connecting, at a substantial mid-point, said arched outer member and said arched inner member, said arched outer member, said arched inner member and said rib member forming first and second cavities,
a retaining pin positioned on a free end of one of said arched outer member and said arched inner member, said retaining pin projecting into an opening of a corresponding one of said first and second cavities; and
a flexible tube, split along a longitudinal axis, said split forming first and second free ends, said first free end slidably engaging said first cavity and said second free end slidably engaging said second cavity, each of said first and second free ends include one or more serrations, said one or more serrations engaging said retaining pin, said flexible tube and said arched outer member having a radius forming said inter vivos tube with a substantially circular cross-section.

12. The inter vivos tube of claim 11, further comprising:
a membrane surrounding said inter vivos tube.

13. The inter vivos tube of claim 11, wherein said retaining pin is one of: fixed and hinged.

14. The inter vivos tube of claim 11, wherein an inner surface of said arched outer member is stepped.

15. The inter vivos tube of claim 11, wherein said arched outer member and said arched inner member are sized to form said cavities to retain said first and second ends to prevent said first and second ends from exiting said corresponding first and second cavities in an expanded mode.

16. An inter vivos tube system comprising:
an expandable inter vivos tube comprising:
a longitudinal H-shaped member comprising:
an arched outer member;
an arched inner member;
a rib member connecting, at a substantial midpoint of said arched outer member and said arched inner member, said arched outer member, said arched inner member and said rib member forming first and second cavities, respectively,
a retaining pin positioned on a free end of one of said arched outer member and said arched inner member, said retaining pin projecting into an opening of a corresponding one of said first and second cavities; and
a flexible tube split along a longitudinal axis, said split forming first and second free ends, said first and second free ends engaging corresponding ones of said first and second cavities, each of said first and second free ends include at least one serration, said at least one serration engaging said retaining pin to retain said first and second free ends at one of a plurality of positions as said first and second free ends are ejected from corresponding first and second cavities, wherein said flexible tube and said arched outer member having a radius forming said inter vivos tube with a substantially circular cross-section; and
an expansion means comprising;
a hollow tube member including a plurality of egress points along a longitudinal axis of said hollow tube member; and
an expandable member attached to a proximate end and to a distal end of said hollow tube member, said hollow tube member sized to fit within an inner diameter of said flexible tube.

17. The system of claim 16, further comprising:
a value for allowing a fluid into said expandable member.

18. The system of claim 17, further comprising:
said value allowing said fluid to be ejected from said expandable member.
